(19) United States
(12) Patent Application Publication
(10) Pub. No.: US 2015/0032040 A1
(43) Pub. Date: Jan. 29, 2015

(54) Garment-Based System, Construction, and Method for Controllably Bracing a Knee

(71) Applicant: Gregory Cadichon, Buffalo Grove, IL (US)

(72) Inventor: Gregory Cadichon, Buffalo Grove, IL (US)

(21) Appl. No.: 13/949,746

(22) Filed: Jul. 24, 2013

(51) Int. Cl. A61F 5/01

(52) U.S. Cl. CPC A61F 5/0123
USPC 602/19

(57) ABSTRACT

A garment-based system, construction and method controls knee brace displacement relative to a brace-outfitted lower extremity. The garment-based system includes or is cooperable with a knee brace, a waist-encircling anchor structure, an anchor-to-brace tether structure, and certain tether-to-brace attachment structures. The waist-encircling anchor structure is receivable about a user's waist and fixable in a transverse anchor plane. The inelastic anchor-to-brace tether structure is fastened to the waist-encircling anchor structure and extends in a first direction orthogonal to the transverse anchor plane. The tether-to-brace attachment structure(s) fasten the knee brace to the anchor-to-brace tether structure, and are immovable in the first direction. The anchor structure, the tether structure, and the attachment structure together function to control knee brace displacement relative to the lower extremity for maintaining the a brace-based pivot axis in proper anatomical alignment with the variable knee axis.

### BACKGROUND OF THE INVENTION

1. Field of the Invention

The present invention generally relates to certain means and methods for bracing a knee. More particularly, the present invention relates to a garment construction arranged so as to operate in combination with a knee brace assembly to prevent and/or control axial displacement of the knee brace assembly relative to an outfitted lower extremity.

2. Discussion of the Prior Art

U.S. Pat. No. 3,932,897, issued to Young, discloses a Hockey Garter Belt. The Young patent describes a hockey garter belt comprising an elongate body encircling member formed from three sections connected end-to-end in which the intermediate section is elasticized so that the belt may be stretched in a body encircling direction. The opposite ends of the belt may be fastened by cooperation of a looped fabric at one end of the belt with hook members fastened to the other end of the belt. A plurality of garter straps is attached to the belt at selected locations to reinforce the belt.

U.S. Pat. No. 4,285,069, issued to Hall, discloses a Garter Belt. The Hall patent describes a garter belt for athletes having a stretchable band extending about the body with its free ends meeting at the front in a shallow upward V. Four straps depend from the forward portion of the band located in pairs on opposite sides of the free end junction. The four straps are each adjustable as to length and there is a fastener on each strap.

U.S. Pat. No. 5,406,942, issued to Loo, discloses a Sacral-Innominate Harness. The Loo patent describes a therapeutic appliance, namely a harness, has a belt, a suspender, a cross-support and an ischial pocket, which harness provides support for the sacroiliac area of a human patient. Use of the harness of this invention will promote resolution of low back pain attributed to sacroiliac strain. The waist belt has two belt ends. The suspender has a strap having two strap ends.

The ischial pocket is stitched on the strap, the ischial pocket forming an ischial receiving area. The cross-support is attached to the strap at an acute angle thereto by a fixed cross-support end of the cross-support, the cross-support also having a free cross-support end. A first fastening means is included on the waist belt for fastening one belt end to the other belt end. A second fastening means is included on the strap for fastening the strap ends onto the belt and a third fastening means is included on the cross-support for fastening the free end thereof to the belt.

U.S. Pat. No. 6,131,206, issued to Vayda, discloses a Garter Belt for Hockey Player. The Vayda patent describes a garter belt for supporting heavy stockings which cover protective leg gear worn by a hockey player. The garter belt includes a preferably elastic elongated waist engaging belt and a plurality of spaced apart garters each connected at an upper end thereof to, and downwardly depend from, the waist belt. Each lower end of each garter includes a first strip of fastener material formed of the hooked portion of two part VELCRO hook and loop fastener.

A second strip of hooked fastener material is connected at its upper end to a mid portion of the garter and positioned to face and be generally coextensive and aligned with the first strip. The first and second strips straddle the stocking opening and are separable for releasable engagement against both inside and outside stocking surfaces just below the stocking opening. At least two garter strips at the front and back of each leg are preferred.

United States Patent Application Publication No. 2006/0293631, authored by Bolt, discloses a Catheter Holder. The Bolt publication describes a catheter urine collection bag with a wide elastic sleeve worn on the upper thigh of the patient. The sleeve is connected by fabric straps to a belt worn around the patient's waist. The fabric straps may be part of a garter belt assembly. A fabric pouch holds the urine collection bag. A portion of the pouch body includes elastic fabric serving holding the bag firmly when filling. The top of the pouch is open to accept the collection bag. Two Velcro tabs on the top of the pouch are positioned to be fastened over the shoulders of the bag to hold it in the pouch. Holes at the bottom of the holder accept the nozzle and valve on the bag that empties the bag as necessary, while a nozzle in the top of the bag is connected to the catheterization tubing of the patient.

United States Patent Application Publication No. 2010/0205721, authored by Ahn discloses a Hose-Holding Undergarment and Corresponding Hose. The Ahn publication describes an improved hose-holding undergarment and corresponding hose equipped with hook-and-loop type of hose-holding devices. The improved hose-holding undergarment is a garter belt, girdle, bustier, or the like with a plurality of hose-holding garter straps each of which has a hose-holding hook of predetermined shape and dimension on the free end of the garter strap.

The corresponding hose has a plurality of strap loops of predetermined shape, dimension, and positions on the upper portion of the hose so that the hose-holding hooks at the free ends of the garter straps can be detachably connected to corresponding strap loops on the top portion of the corresponding hose. Compared to the conventional button-and-buttonhole type of hose-holding devices, the hook-and-loop type of hose-holding device of the present invention is simpler in structure, thinner in thickness, lighter in weight, surer in hose-gripping action, and easier to use.

United States Patent Application Publication No. 2011/0197335, authored by Handy, discloses a Combination Shirt and Suspenders. The Handy publication describes a combination shirt and suspender construction comprising an undershirt or t-shirt together with a pair of suspender straps. The suspender straps are secured to the underlying shirt surface by sewn seams. Each suspender strap further supports an end strap having a fabric attachment portion thereon. The user wears the shirt in a conventional manner and secures the suspender straps to a pants belt or waistband to provide suspender support of the wearer's pants.

### SUMMARY OF THE INVENTION

All braces whether functional braces for ACL or PCL deficiency, collateral ligament injury, OA braces (for Osteoarthritis), prophylactic braces for prevention of ligament injury, or post-operative knee braces have one hinge or two hinges on either side of the knee joint. This hinge is either uniaxial or polyaxial in design. The polyaxial design has been implemented due to the difficulty in reproducing with a hinge the motion of the knee joint, which is a “rolling hinge” and creates a moving instant center of rotation in a semicircular line. All hinges of any type need to be placed directly over that semicircular line (near the anatomical femoral epicondyle) to reproduce knee flexion-extension biomechanics as much as possible.

A review of the literature yields an article by P. S. Walker et al. in the 1988 Journal of Biomechanics (Vol. 21 pp. 965-974) in which it is stated that their results indicated that a 5 mm hinge displacement created large changes in knee mechanics. This research was later utilized by Bruce Brownstein (in his article from the Journal of Sport Rehabilitation 1988 Vol. 7 pp. 33-43) in which he compared 14 different functional knee braces and measured brace displacement (migration). Brownstein used the criterion of 5 mm or less of brace migration as acceptable. Brownstein went on to say that significant brace migration is unacceptable insofar as it tends moves the hinge and reduces the effectiveness of the knee brace, and it puts forces on the knee that risk injury.

To control knee brace migration or displacement of the hinge off the center of rotation of the knee, the present invention provides a garment construction for controlling knee brace displacements relative to a brace-outfitted lower extremity. The garment construction according to the present invention preferably and essentially comprises a waist-encircling anchor structure, an anchor-to-brace tether structure, and certain tether-to-brace attachment means.

The waist-encircling anchor structure is received about a user's waist and selectively fixed in a transverse anchor plane at or about the user's waist. The anchor-to-brace tether structure is fastened to the waist-encircling anchor structure and extends in a first direction substantially orthogonal to the transverse anchor plane and parallel to the lower extremity axis. The anchor-to-brace tether structure is preferably inelastic in the first direction.

The tether-to-brace attachment means as preferably exemplified hereinabove essentially fasten an extremity-outfitted knee brace to the anchor-to-brace tether structure. The tether-to-brace attachment means are preferably immovable in the first direction. Accordingly, the waist-encircling anchor structure, the anchor-to-brace tether structure, and the tether-to-brace attachment means effectively and cooperably function to control (axial) displacement(s) of the extremity-outfitted knee brace relative to a brace-outfitted lower extremity.

The preferred garment construction according to the present invention preferably further comprises a pant construction, which pant construction is cooperably fastened to the waist-encircling anchor structure, the anchor-to-brace tether structure, and the tether-to-brace attachment means for further enhancing control of knee brace displacement(s). The pant construction preferably comprises or consists of an elastic material and fastens the anchor-to-brace tether structure to the tether-to-brace attachment means, such that the elastic material enables a range of brace-to-extremity displacement on the order of +/−5 mm generally within the plane of the material for properly aligning the tethered knee brace with a variable flexion-extension axis of the lower extremity.

In addition to the structural considerations of the garment construction set forth hereinafter, it is further contemplated that the structural bases support a certain garment-implemented knee bracing method for controlling (axial) displacement(s) of a knee brace relative to a brace-outfitted lower extremity. The garment-implemented method(s) according to the present invention may be said to comprise the initial step of providing a garment construction, the garment construction comprising a waist-encircling anchor structure, an anchor-to-brace tether structure, and certain tether-to-brace attachment means substantially as previously specified.

The waist-encircling anchor structure may be encircled about a user's waist, and fixed thereabouts in a transverse anchor plane. The target lower extremity may then be outfitted with a knee brace thereby forming an extremity-outfitted knee brace, which knee brace necessarily comprises at least one knee brace axis. The knee brace is then tethered to the waist-encircling anchor structure via the anchor-to-brace tether structure and tether-to-brace attachment means. Displacements of the extremity-outfitted knee brace relative to the user's lower extremity may thus be controlled via the waist-encircling anchor structure, the anchor-to-brace tether structure, and the tether-to-brace attachment means.

The knee bracing method may further preferably comprise the steps of extending the anchor-to-brace tether structure from an anterior portion of the waist-encircling anchor structure along an anterior portion of the lower extremity and fastening the anchor-to-brace tether structure to an anterior, thigh-traversing member of the knee brace via the tether-to-brace attachment means, thereby anteriorly tethering the knee brace to the waist-encircling anchor structure.

Bearing mind the preferred garment construction 10, the knee bracing method of may further preferably comprises the step of fastening the waist-encircling anchor structure, the anchor-to-brace tether structure, and the tether-to-brace attachment means to an elastic pant construction for further enhancing control of knee brace displacements. The elastic pant construction may extend in a layer intermediate the anchor-to-brace tether structure and the tether-to-brace attachment means for elastically fastening the anchor-to-brace tether structure to the tether-to-brace attachment means, thereby restricting a range of brace-to-extremity axial displacement via the elastically fastened anchor-to-brace tether structure and tether-to-brace attachment means. Anatomical alignment of the at least one knee brace axis may thus be maintained with the variable flexion-extension axis of the outfitted lower extremity within said range of brace-to-extremity displacement.

### BRIEF DESCRIPTION OF THE DRAWINGS

Other features of my invention will become more evident from a consideration of the following brief descriptions of drawings:

FIG. 1 is an anterior perspective view of a preferred garment construction according to the present invention shown attached to a lower-extremity outfitted knee brace.

FIG. 2 is an enlarged boxed view of a layered arrangement of materials juxtaposed atop a user's lower extremity, showing from top to bottom a brace construction, a first matable hook structure, a second matable loop structure, a pant layer, a tether layer, and a skin layer.

FIG. 3 is an enlarged fragmentary sectional view in a first dimension of the lower portion of the garment construction according to the present invention depicting areas of greater versus lesser elasticity of the materials.

FIG. 4 is an enlarged fragmentary sectional view in a second dimension of the lower portion of the garment construction according to the present invention depicting areas of greater versus lesser elasticity of the materials.

FIG. 5 is an anterior view of a preferred garment construction according to the present invention as outfitted upon a user.

FIG. 6 is a posterior view of the preferred garment construction according to the present invention as outfitted upon a user.

FIG. 7 is an anterior view of an alternative garment construction according to the present invention as outfitted upon a user.

FIG. 8 is a posterior view of the alternative garment construction according to the present invention as outfitted upon a user.

FIG. 9 is a lateral view of a fragmentary femur-to-tibia or knee junction site depicting a lower extremity axis and joint plane orthogonal to the lower extremity axis with the femur and tibia being shown in axial alignment with the lower extremity axis.

FIG. 10 is a lateral view of the fragmentary femur-to-tibia or knee junction site depicting a lower extremity axis and joint plane orthogonal to the lower extremity axis with the femur being shown oblique to the lower extremity axis.

FIG. 11 is a first fragmentary anterior perspective view of a knee joint of a lower extremity outfitted with a knee brace with parts of the lower extremity being removed to show the femur, tibia, and patella relative to the knee brace.

FIG. 12 is a fragmentary anterior view of the fragmentary femur-to-tibia or knee junction site.

FIG. 13 is a second fragmentary anterior perspective view of a knee joint of a lower extremity outfitted with a knee brace with parts of the lower extremity being removed to show the femur, tibia, and patella relative to the knee brace, and depicting a permissible or preferred range of axial migration for the knee brace.

FIG. 14 is a diagrammatic depiction of the various planes defined by the garment construction according to the present invention juxtaposed adjacent a generic knee brace to generally and generically depict how knee brace displacements are controlled according to the present invention.

### DETAILED DESCRIPTION OF THE PREFERRED EMBODIMENT

Referring now the drawings with more specificity, the present invention essentially provides a garment-based system or construction 10 for generally controlling axial displacements of a knee brace 11 relative to a brace-outfitted lower extremity as at 100, and for more particularly for enabling slightly varied pivot axis movement or migration at or about the variable flexion-extension (i.e. FE) axis of a human knee joint so as to more properly maintain the pivotal knee brace in anatomical alignment with the variable FE axis.

Maintaining a knee brace in proper outfitted position upon a user's lower extremity 100 can often be difficult particularly if the lower extremity 100 engages in a great deal of activity. Accelerations and vibrations can quickly cause a knee brace outfitted upon a lower extremity 100 to displace from its proper axial position relative to the axis 105 of the lower extremity 100.

While straps 12 and other brace positioning means do provide some acceptable level of proper position securement, these brace positioning means often lack the ability to permanently fix the brace in proper position. Further state of the art bracing means and brace-positioning means do not enable a user to fix the proper brace position and simultaneously provide certain means for dynamically stabilizing the axis or axes of the knee brace to anatomically correspond with naturally occurring variances in knee joint anatomy during flexion-extension. As prefatorily stated hereinabove, all braces whether functional braces for Anterior Cruciate Ligament (ACL 107) injury, Posterior Cruciate Ligament (PCL 108) injury, Collateral Ligament injury, Osteoarthritis (OA) braces, Prophylactic braces for prevention of ligament injury, or post-operative knee braces have certain hinge means incorporated into the design. These hinge means can be either uniaxial or polyaxial in design.

The polyaxial designs have been implemented largely as a means to address the structural difficulty in reproducing with a hinge the naturally occurring anatomical motion of the knee joint, which may be described as “rolling hinge”, which rolling hinge creates a moving instant center of rotation (as at 101) in a semicircular line. State of the art knee braces do not adequately mimic the naturally occurring rolling hinge of the knee joint, and by default may incorporate structurally complex polyaxial hinge means in an attempt to more properly follow the naturally occurring knee joint motion during flexion-extension.

All hinges of any type, are generally designed for positioned placement adjacent the semicircular line of the instant center of rotation of the knee near the anatomical femoral epicondyle structure(s)) to reproduce knee flexion-extension biomechanics as much as possible. Walker et al. have reported that a 5 millimeter (mm) hinge displacement created large changes in knee mechanics. This research was further cited by Brownstein in his research in which he compared 14 different braces and measured brace migration.

Walker et al. and Brownstein have thus used the criterion of 5 mm brace and thus hinge axis migration for a knee brace as acceptable. Providing a knee brace that allows for this minimal amount of hinge-axis migration has proved difficult, however. The present invention attempts to eliminate gross brace (and thus hinge axis) migration indirectly by way of a garment-based system or construction, which system operates in combination with any number of state of the art knee brace assemblies to yield an overall system that maintains the knee brace in proper axial position relative to the axis 105 of the lower extremity 100, yet prevents gross brace axis migration thus more properly maintaining the naturally occurring rolling hinge movement of the human knee joint.

As noted, a lower extremity 100 of a human inherently has a variable, knee-based flexion-extension (FE) axis having a moving instant center of rotation as at 101, which center of rotation 101 is perpendicular to the sagittal plane dividing the human form into left and right halves. The tibia 103 does have some degree of rotation as at 109 about the axis 105 and the center of rotation 110 for the tibia 103 which rotation is also structurally controlled to some degree by the knee bracing systems generally.

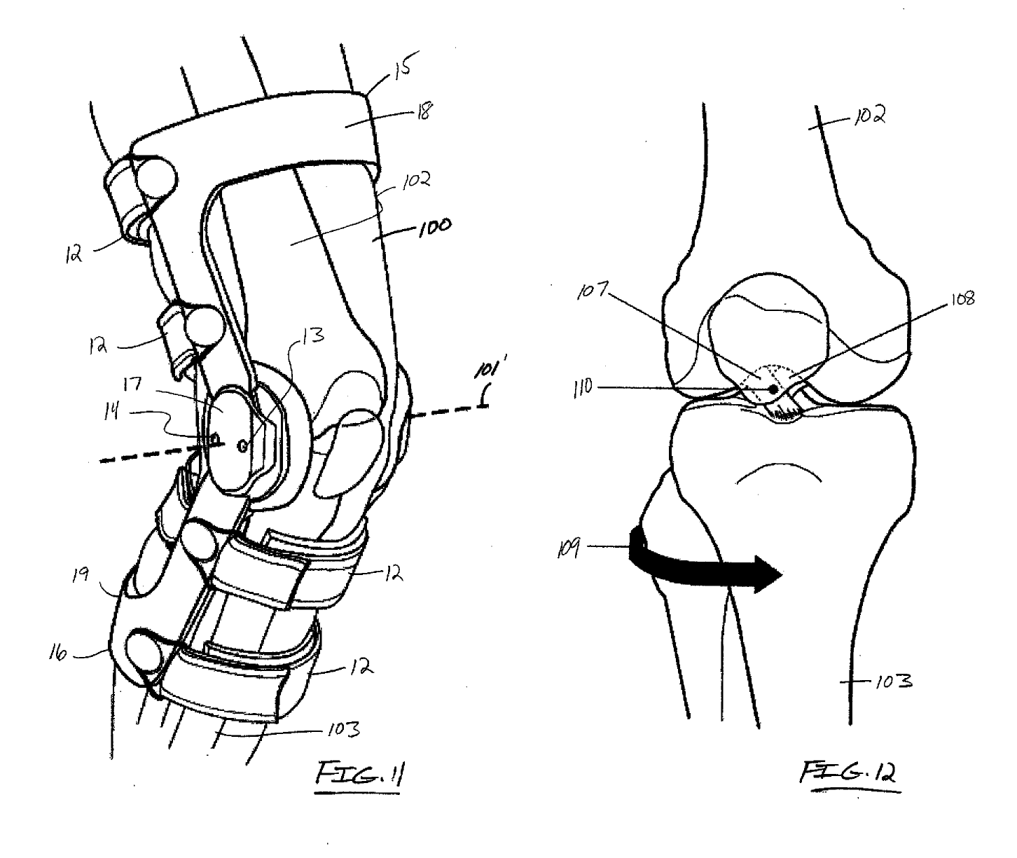

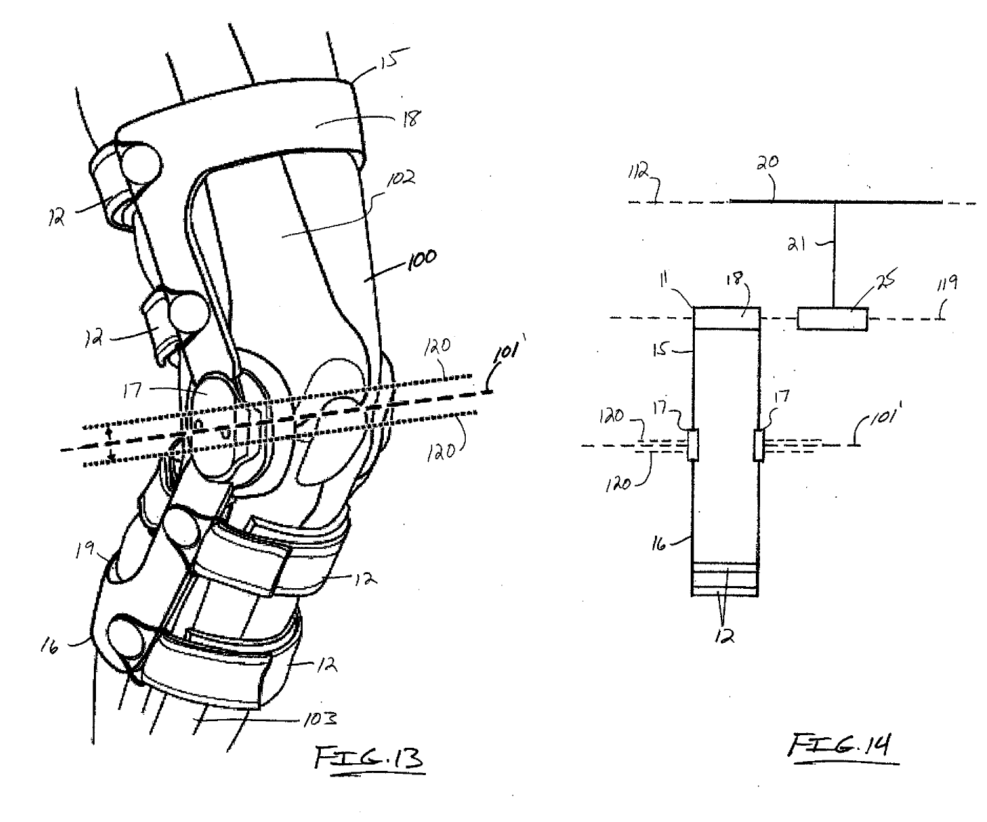

In this regard, the reader will note that the moving instant center of rotation 101 is depicted as circular having a diameter in contradistinction to a fixed axis which in two dimensions would be depicted as a point (the axis extending into/out of the page). In this case, the diameter of the instant center of rotation 101 embraces the boundaries in which the knee joint axis may be found at any point during the flexion-extension activity. An axis 101′ is depicted in FIG. 11 as defining the center point or axis of the center of rotation 101.

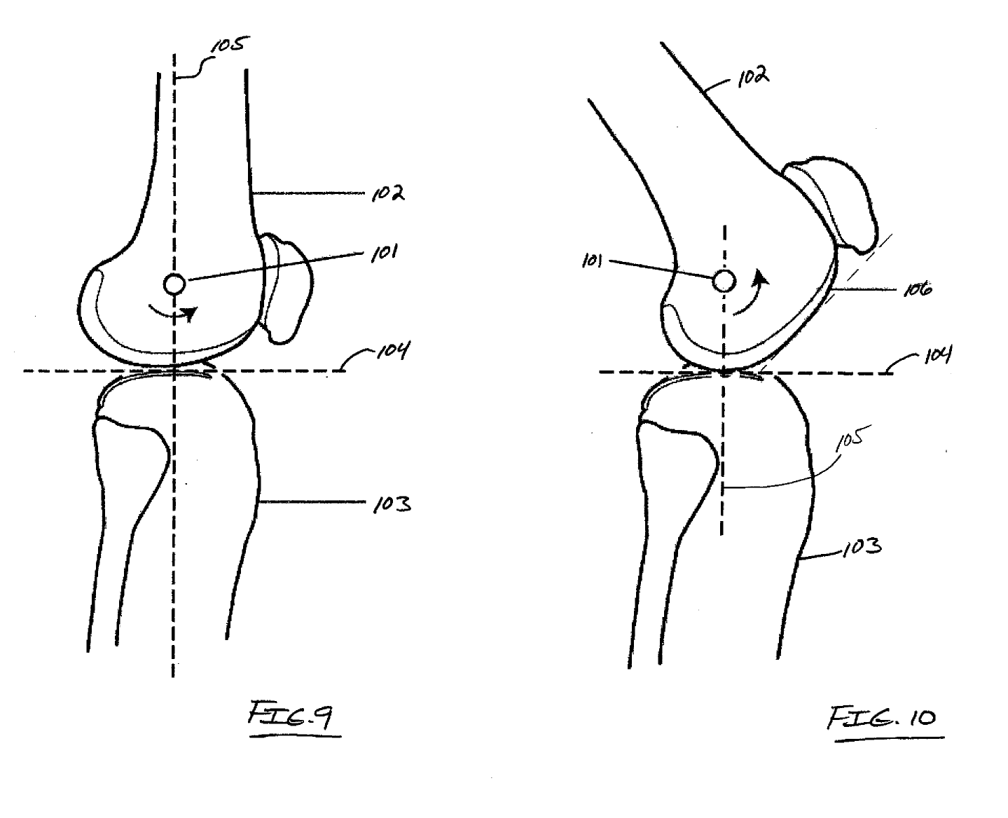

The upper femur 102 and the lower tibia 103 meet at the knee joint or junction and are separated by a joint line or plane as at 104. Together, the femur 102 and the tibia 103 may be said to define a lower extremity axis as at 105 when the lower extremity 100 is held straight at the knee joint or junction site. During a bending movement or action, the joint surface 106 of the femur 102 translates from a joint line-to-surface (substantially) parallel configuration as generally depicted in FIG. 9 to a joint line-to-surface oblique configuration as generally depicted in FIG. 10.

The knee brace 11 depicted in the drawings accompanying these specifications is presented as an exemplary knee brace, and not meant to limit the invention. Stated another way, the knee brace illustrated and referenced at 11 is a polycentric or polyaxial knee brace having an anterior axis point at 13 and a posterior axis point at 14. Other polyaxial knee braces in the art show proximal axis points juxtaposed distal axis points or combinations of three axis points. Still further, some knee braces provide a uni-axis structure, the latter of which tend to be disfavored for many of the reasons stated hereinabove.

The knee brace 11 cooperable with the garment construction 10 according to the present invention is believed to comprise certain essential features, including a thigh-engaging upper brace assembly as at 15, a calf-engaging lower brace assembly as at 16, and certain hinge means for pivotally attaching the upper brace assembly 15 to the lower brace assembly 16. The hinge means of the knee brace 11 according to or cooperable with the present invention preferably comprises at least one brace-based pivot axis of rotation enabled or provided by laterally opposed upper to lower brace assembly junction structures 17.

The upper brace assembly 15 preferably comprises an anterior, thigh-traversing member or portion as at 18, and the lower brace assembly 16 preferably comprises a posterior, calf-traversing member as at 19. Straps 12 are fastened to the upper brace assembly 15 extending posterior to the thigh for transversely securing the upper brace assembly 15 to the thigh portion of the lower extremity 100, and straps 12 are fastened to the lower brace assembly 16 extending anterior to the lower leg for transversely securing the lower brace assembly 16 to the lower leg portion of the lower extremity 100.

Notably, accelerations and vibrations in the direction of axis 105 tend to displace the knee brace 11 relative to the axis 105 when the primary means for securing the knee brace 11 to the lower extremity 100 is by way of transverse or extremity-encircling straps 12. In order to prevent these undesirable axial displacements, the garment construction 10 according to the present invention preferably comprises a waist-encircling anchor structure as at 20, an anchor-to-brace tether structure as at 21, certain tether-to-brace attachment means, as may be exemplified by matable VELCRO® brand hook and loop type fastening structure, and a pant construction as at 22.

As indicated, the preferred tether-to-brace attachment means may be defined by first and second select hook-loop fastening structures. A first select hook-loop fastening structure 25 of the matable hook and loop type fastening structures is selectively matable with a second select hook-loop fastening structure 26 of the matable hook and loop type fastening structures.

The first select hook-loop fastening structure 25 is outfitted upon or fixedly attached to the anchor-to-brace tether structure 21, and the second select hook-loop fastening structure 26 is outfitted upon or fixedly attached to the undersurface 24 of the thigh-traversing member or portion 18 of the upper brace assembly 15. The first and second select hook-loop fastening structures are preferably interchangeably selected from the group consisting of hook type fastening structure as at 27 and loop type fastening structure as at 28.

The waist-encircling anchor structure 20 is preferably receivable about a user's waist 111 and fixable in a transverse anchor plane as at 112 about the user's waist 111. The waist-encircling anchor structure 20 comprises a reinforced waist band 23 and a draw string as at 29. Together, the reinforced waist band 23 and the draw string 29 provide the user with certain means for tightening the anchor structure 20 about the waist 111 to enhance fixation of the transverse anchor plane 112. In other words, the primary function of the anchor structure 20 is to provide an anchor or fixed plane 112, and the reinforced waist band 23 and draw string 29 provide the user with means for providing the fixed plane 112.

The anchor-to-brace tether structure 21 is preferably fastened (e.g. stitched as at 30) to the waist-encircling anchor structure 20 and extends in a first direction substantially parallel to the lower extremity axis 105 and substantially orthogonal to the transverse anchor plane 112. The anchor-to-brace tether structure 21 is necessarily inelastic in the first direction. The anchor-to-brace tether structure 21 preferably extends from an anterior portion of the waist-encircling anchor structure 20, and is fastened to the anterior, thigh-traversing member or portion 18 via the tether-to-brace attachment means for anteriorly tethering the knee brace 11 to the waist-encircling anchor structure 20 (in the fixed transverse plane 112).

The tether-to-brace attachment means primarily function to fasten the anterior thigh-traversing member or portion 18 to a lower end of the anchor-to-brace tether structure 21. The tether-to-brace attachment means are preferably inelastic or immovable in the first direction. Accordingly, the waist-encircling anchor structure 20, the anchor-to-brace tether structure 21, and the tether-to-brace attachment means together cooperate to control axial displacements of the knee brace 11 relative to the lower extremity 100 for maintaining the at least one brace-based pivot axis in proper anatomical alignment with the variable FE axis of rotation defined or embraced by the center of rotation 101.

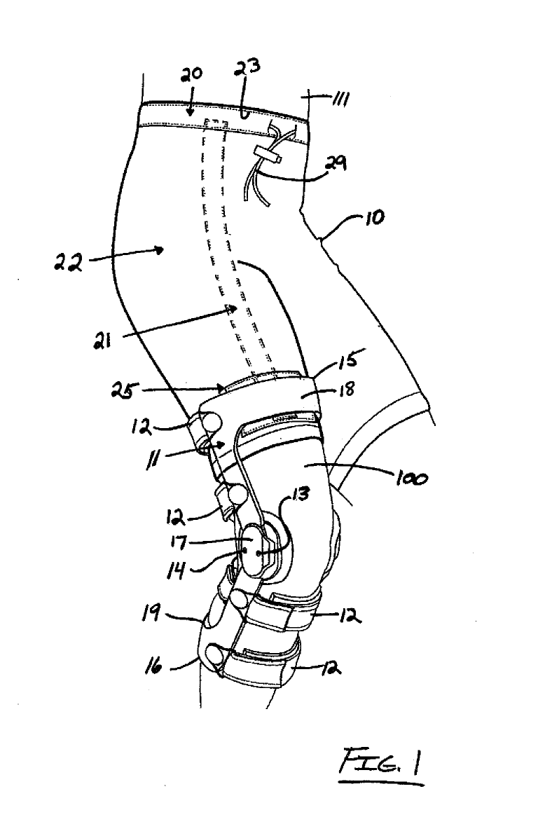

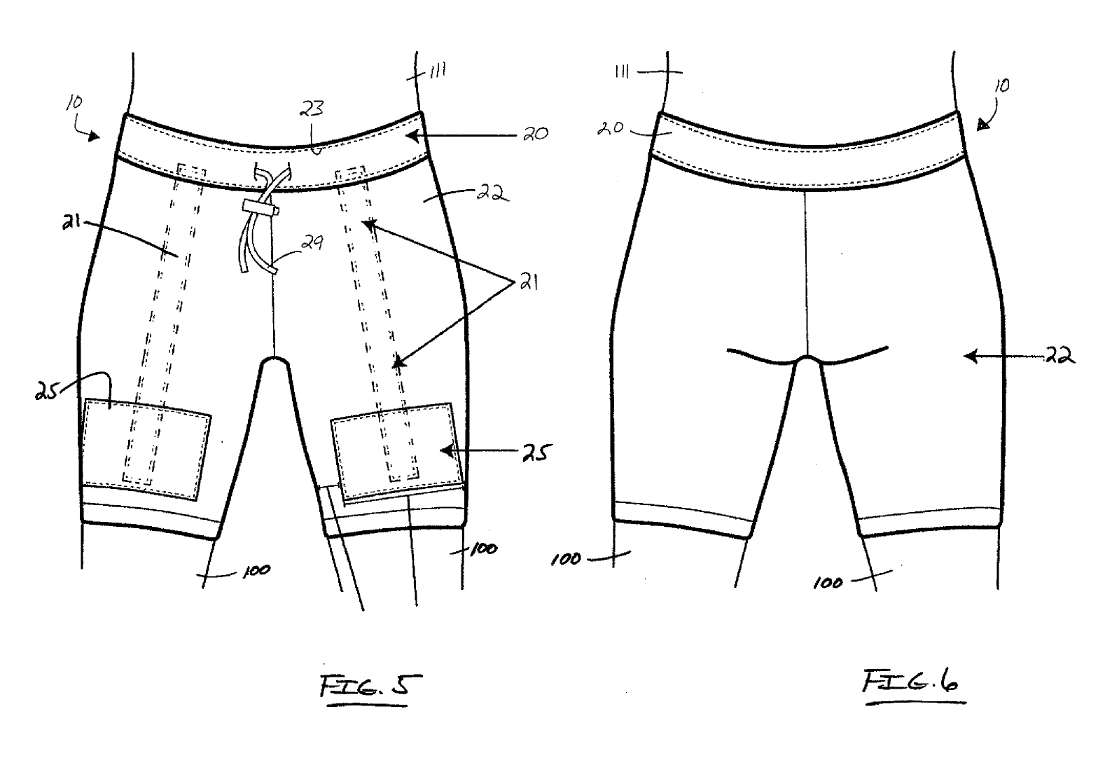

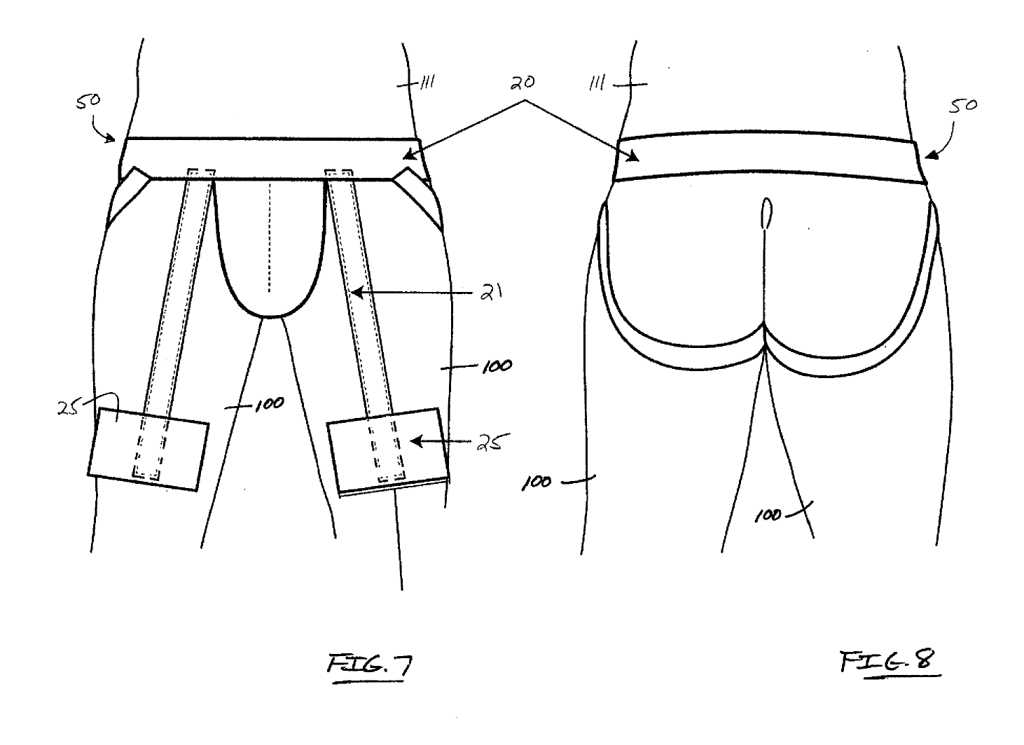

The incorporation or addition of the pant construction 22 into garment construction 10 essentially distinguishes the preferred garment construction 10 depicted in FIGS. 1, 5, and 6 from the alternative garment construction 50 depicted in FIGS. 7 and 8. The pant construction covers the waist-encircling anchor structure 20 and the anchor-to-brace tether structure 21 and is cooperably fastened to the waist-encircling anchor structure 20, the anchor-to-brace tether structure 21, and the tether-to-brace attachment means for enhancing control of knee brace axial displacement(s).

In this regard, the pant construction 22 preferably comprises or consists of an elastic material such as spandex or elastane type fabric material, and preferably extends in a layer intermediate the anchor-to-brace tether structure 21 and the tether-to-brace attachment means for elastically fastening the anchor-to-brace tether structure 21 to the tether-to-brace attachment means. The elastic material of the pant construction 22 situated intermediate the anchor-to-brace tether structure 21 and the tether-to-brace attachment means enables a range of brace-to-extremity displacement(s).

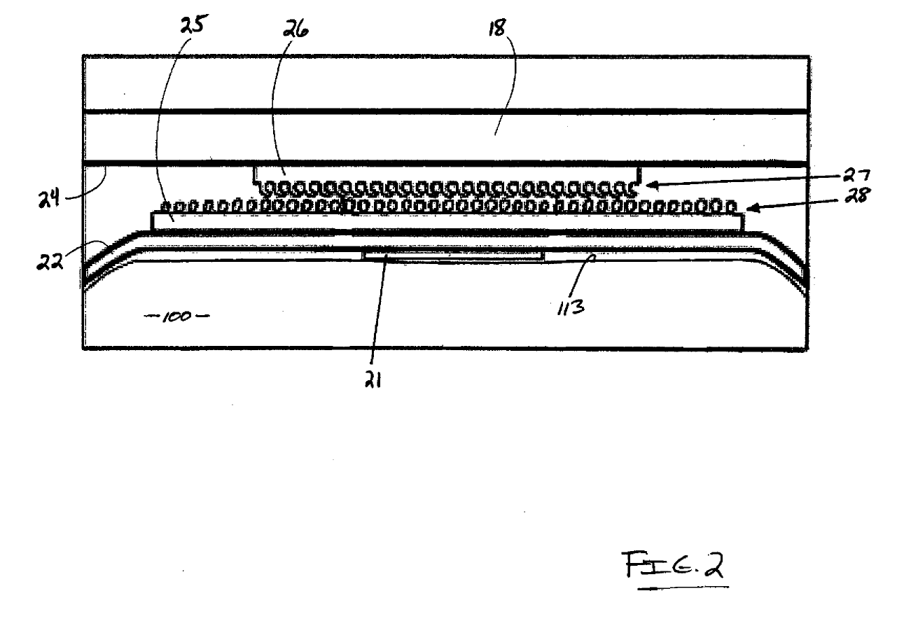

The reader is directed to FIGS. 2-4, which figures depict various layers of the garment construction 10 relative to the knee brace 11. FIG. 2 is a boxed view of various layers starting from bottom to top, a skin layer 113 of the lower extremity 100, the anchor-to-brace tether structure 21, the pant construction 22, the second select hook-loop fastening structure 26, the first select hook-loop fastening structure 25, and the anterior thigh-traversing member or portion 18 of the upper brace assembly 15.

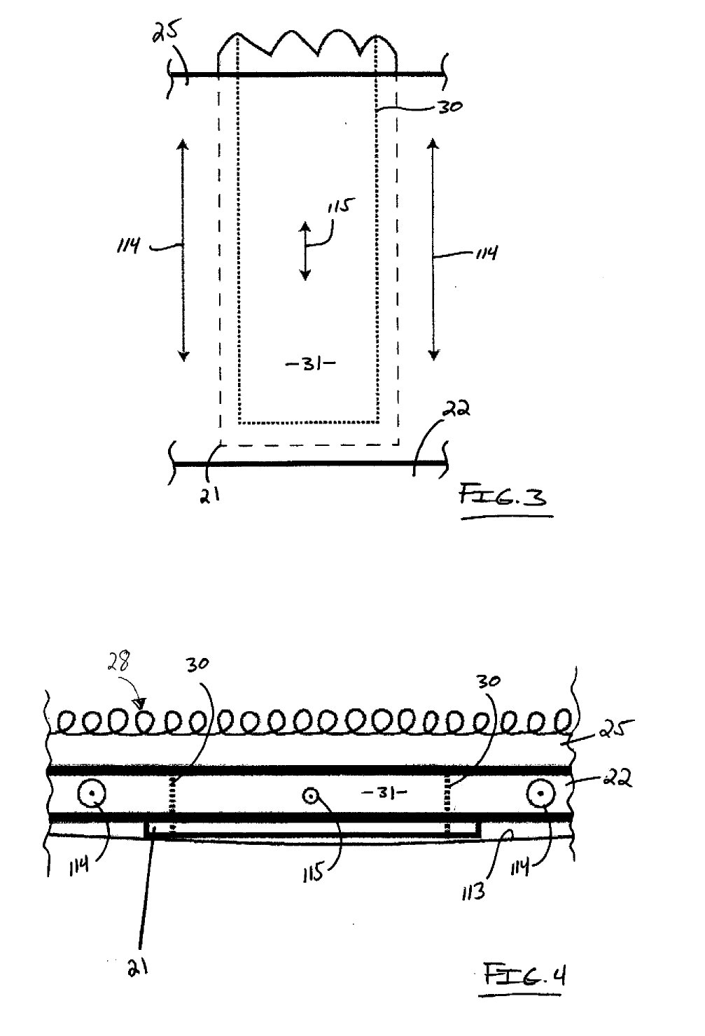

Comparatively referencing FIGS. 3 and 4, the reader will note vectors 114 and 115. Vectors 114 lie within the upper and lower boundary layers of the pant construction 22 and represent unrestricted elasticity of the material construction. Vectors 114 are relatively greater in length and magnitude than the vector(s) 115, which vector(s) 115 depict restricted or restrained elasticity within the area bound by stitching 30 where the anchor-to-brace tether structure is stitched or otherwise fastened to the pant construction 22. This area may be defined as a tether-to-pant junction region 31.

The range of brace-to-extremity displacements enabled within the material layer of the tether-to-pant junction region 31 are preferably +/−5 mm (as at boundaries 120) as per the research and reporting of Walker et al. and Brownstein for anatomically, properly, and correspondingly aligning the at least one pivot axis of the knee brace 11 with the variable flexion-extension (FE) axis or center or rotation 101 of the knee joint or junction. In other words, the tether-to-pant junction region 31 defines an area of restrained elasticity having reduced elasticity relative to the elastic material of the pant construction 22 for providing axial migration(s) during flexion-extension activity.

Referencing FIG. 14, the reader will thus understand that the fixed anchor plane 112 serves as a foundation from which axial displacement control can be effected. The inelastic anchor-to-brace tether structure 21 inelastically positions the first select hook-loop fastening structure 25 at a distance or plane 119 from the transverse plane 112. The second select hook-loop fastening structure 26 (attached to the back or underside of member 18) mates with the structure 25 and immovably maintains the upper brace assembly 15 at the plane 19.

The primary structures of garment constructions 10 and 50 axially position the knee brace 11 such that the (at least one) brace-based pivot axis may be anatomically and/or axially aligned with the variable flexion-extension axis 101′ of the lower extremity 100. The garment construction 10 includes an elastic medium (i.e. the pant construction 22) which operates to enhance the variability of axial migration as generically depicted by the boundaries 120.

While the foregoing specifications set forth much specificity, the same should not be construed as setting forth limits to the invention but rather as setting forth certain preferred embodiments and features. For example, it is contemplated that the foregoing specifications support a garment construction for controlling knee brace displacements relative to a brace-outfitted lower extremity. The garment construction according to the present invention preferably and essentially comprises a waist-encircling anchor structure as at 20, an anchor-to-brace tether structure as at 21, and certain tether-to-brace attachment means as believably embraced by both garment construction 10 (e.g. a shorts-based or type garment construction) and garment construction 50 (e.g. an athletic supporter based or type garment construction).

The waist-encircling anchor structure 20 is received about a user's waist and selectively fixed in a transverse anchor plane at or about the user's waist. The anchor-to-brace tether structure 21 is fastened to the waist-encircling anchor structure 20 and extends in a first direction substantially orthogonal to the transverse anchor plane and parallel to the lower extremity axis 105. The anchor-to-brace tether structure 21 is preferably inelastic in the first direction.

The tether-to-brace attachment means as preferably exemplified hereinabove essentially fasten an extremity-outfitted knee brace to the anchor-to-brace tether structure 21. The tether-to-brace attachment means are preferably immovable in the first direction. Accordingly, the waist-encircling anchor structure 20, the anchor-to-brace tether structure 21, and the tether-to-brace attachment means effectively and cooperably function to control (axial) displacement(s) of the extremity-outfitted knee brace 11 relative to a brace-outfitted lower extremity 100.

The garment construction 10 according to the present invention preferably further comprises a pant construction as at 22, which pant construction is cooperably fastened to the waist-encircling anchor structure 20, the anchor-to-brace tether structure 21, and the tether-to-brace attachment means for enhancing control of knee brace displacement(s). The pant construction 22 preferably comprises or consists of an elastic material and fastens the anchor-to-brace tether structure 21 to the tether-to-brace attachment means, such that the elastic material enables a range of brace-to-extremity displacement on the order of +/−5 mm generally within the plane of the material for properly aligning the tethered knee brace with a variable flexion-extension axis of the lower extremity.

In addition to the foregoing structural considerations, it is further contemplated that the structural bases support a certain garment-implemented knee bracing method for controlling (axial) displacement(s) of a knee brace relative to a brace-outfitted lower extremity. The garment-implemented method(s) according to the present invention may be said to comprise the initial step of providing a garment construction, the garment construction comprising a waist-encircling anchor structure, an anchor-to-brace tether structure, and certain tether-to-brace attachment means substantially as previously specified.

The waist-encircling anchor structure may be encircled about a user's waist, and fixed thereabouts in a transverse anchor plane. The target lower extremity may then be outfitted with a knee brace thereby forming an extremity-outfitted knee brace, which knee brace necessarily comprises at least one knee brace axis. The knee brace is then tethered to the waist-encircling anchor structure via the anchor-to-brace tether structure and tether-to-brace attachment means. Displacements of the extremity-outfitted knee brace relative to the user's lower extremity may thus be controlled via the waist-encircling anchor structure, the anchor-to-brace tether structure, and the tether-to-brace attachment means.

The knee bracing method may further preferably comprise the steps of extending the anchor-to-brace tether structure from an anterior portion of the waist-encircling anchor structure along an anterior portion of the lower extremity and fastening the anchor-to-brace tether structure to an anterior, thigh-traversing member of the knee brace via the tether-to-brace attachment means, thereby anteriorly tethering the knee brace to the waist-encircling anchor structure.

Bearing mind the preferred garment construction 10, the knee bracing method of may further preferably comprises the step of fastening the waist-encircling anchor structure, the anchor-to-brace tether structure, and the tether-to-brace attachment means to an elastic pant construction for further enhancing control of knee brace displacements.

The elastic pant construction may extend in a layer intermediate the anchor-to-brace tether structure and the tether-to-brace attachment means for elastically fastening the anchor-to-brace tether structure to the tether-to-brace attachment means, thereby enabling a restricted or restrained range of brace-to-extremity axial displacement via the elastically fastened anchor-to-brace tether structure and tether-to-brace attachment means. Anatomical alignment of the at least one knee brace axis may thus be maintained with the variable flexion-extension axis of the outfitted lower extremity within said range of brace-to-extremity displacement.

Accordingly, although the invention has been described by reference to certain preferred and alternative embodiments, and certain novel knee bracing methodology, it is not intended that the novel arrangements be limited thereby, but that modifications thereof are intended to be included as falling within the broad scope and spirit of the foregoing disclosures and the appended drawings.

I claim:

1. A garment-based system for controlling knee brace displacement relative to a brace-outfitted lower extremity, the lower extremity having a knee-based variable flexion-extension axis, the garment-based system comprising, in combination:
a knee brace, the knee brace comprising a thigh-engaging upper brace assembly, a calf-engaging lower brace assembly, and means for pivotally attaching the upper brace assembly to the lower brace assembly, said means comprising at least one brace-based pivot axis;
a waist-encircling anchor structure, the waist-encircling anchor structure being receivable about a user's waist and fixable in a transverse anchor plane;
an anchor-to-brace tether structure, the anchor-to-brace tether structure being fastened to the waist-encircling anchor structure and extending in a first direction, the first direction being substantially orthogonal to the transverse anchor plane and parallel to a lower extremity axis, the anchor-to-brace tether structure being inelastic in the first direction; and
tether-to-brace attachment means, the tether-to-brace attachment means for fastening the upper brace assembly to the anchor-to-brace tether structure, the tether-to-brace attachment means being immovable in the first direction, the waist-encircling anchor structure, the anchor-to-brace tether structure, and the tether-to-brace attachment means thus for controlling knee brace displacement relative to the lower extremity for maintaining the at least one brace-based pivot axis in proper anatomical alignment with the variable flexion-extension axis.

2. The garment-based system of claim 2 comprising a pant construction, the pant construction covering the waist-encircling anchor structure and anchor-to-brace tether structure and being cooperably fastened to the waist-encircling anchor structure, the anchor-to-brace tether structure, and the tether-to-brace attachment means for enhancing control of said knee brace displacement.

3. The garment-based system of claim 2 wherein the pant construction comprises an elastic material, the pant construction extending in a layer intermediate the anchor-to-brace tether structure and the tether-to-brace attachment means for elastically fastening the anchor-to-brace tether structure to the tether-to-brace attachment means, the elastic material of the pant construction enabling a range of brace-to-extremity displacement, the range of brace-to-extremity displacement for anatomically, properly, and correspondingly aligning the at least one brace-based pivot axis with the variable flexion-extension axis.

4. The garment-based system of claim 3 wherein the upper brace assembly comprises an anterior, thigh-traversing member, the anchor-to-brace tether structure extending from an anterior portion of the waist-encircling anchor structure and being fastened to the anterior, thigh-traversing member via the tether-to-brace attachment means for anteriorly tethering the knee brace to the waist-encircling anchor structure.

5. The garment-based system of claim 3 wherein the waist-encircling anchor structure comprises a reinforced waist band and a draw string, the reinforced waist band and draw string for enhancing fixation of the transverse anchor plane.

6. The garment-based system of claim 3 comprising a tether-to-pant junction region, the tether-to-pant junction region defining an area of restrained elasticity, the area of restrained elasticity having reduced elasticity relative to the elastic material of the pant construction.

7. The garment-based system of claim 4 wherein the tether-to-brace attachment means are defined by a first select hook-loop fastening structure, the first select hook-loop fastening structure being selectively matable with a second select hook-loop fastening structure outfitted upon the anterior, thigh-traversing member, the first and second select hook-loop fastening structures being selected from the group consisting of hook type fastening structure and loop type fastening structure.

8. A garment construction for controlling knee brace displacement relative to a brace-outfitted lower extremity, the garment construction comprising:
a waist-encircling anchor structure, the waist-encircling anchor structure being receivable about a user's waist and fixable in a transverse anchor plane;
an anchor-to-brace tether structure, the anchor-to-brace tether structure being fastened to the waist-encircling anchor structure and extending in a first direction, the anchor-to-brace tether structure being inelastic in the first direction; and
tether-to-brace attachment means, the tether-to-brace attachment means for fastening an extremity-outfitted knee brace to the anchor-to-brace tether structure, the tether-to-brace attachment means being immovable in the first direction, the waist-encircling anchor structure, the anchor-to-brace tether structure, and the tether-to-brace attachment means thus for controlling knee brace displacement relative to a brace-outfitted lower extremity.

9. The garment construction of claim 8 comprising a pant construction, the pant construction being cooperably fastened to the waist-encircling anchor structure, the anchor-to-brace tether structure, and the tether-to-brace attachment means for enhancing control of said knee brace displacement.

10. The garment construction of claim 9 wherein the pant construction comprises an elastic material, the pant construction fastening the anchor-to-brace tether structure to the tether-to-brace attachment means, the elastic material of the pant construction enabling a range of brace-to-extremity displacement, the range of brace-to-extremity displacement for properly aligning a brace-based pivot axis with a variable flexion-extension axis.

11. The garment construction of claim 10 wherein the waist-encircling anchor structure comprises a reinforced waist band and a draw string, the reinforced waist band and draw string for selectively enhancing fixation of the transverse anchor plane.

12. The garment construction of claim 10 comprising a tether-to-pant junction region, the tether-to-pant junction region defining an area of restrained elasticity, the area of restrained elasticity having reduced elasticity relative to the elastic material of the pant construction.

13. The garment construction of claim 10 wherein the tether-to-brace attachment means are defined by a first select hook-loop fastening structure, the first select hook-loop fastening structure being selectively matable with a second select hook-loop fastening structure outfitted upon the extremity-outfitted knee brace, the first and second select hook-loop fastening structures being selected from the group consisting of hook type fastening structure and loop type fastening structure.

14. A garment-implemented knee bracing method for controlling knee brace displacement relative to a brace-outfitted lower extremity, the garment-implemented knee bracing method comprising the steps of:
providing a garment construction, the garment construction comprising an anchor structure, an anchor-to-brace tether structure, and tether-to-brace attachment means;
fixing the anchor structure in a transverse anchor plane;
outfitting a knee brace upon a user's lower extremity, thereby forming an extremity-outfitted knee brace, the knee brace having a knee brace axis;
tethering the knee brace to the anchor structure via the anchor-to-brace tether structure and tether-to-brace attachment means; and
controlling knee brace displacement of the extremity-outfitted knee brace relative to the user's lower extremity via the anchor structure, the anchor-to-brace tether structure, and the tether-to-brace attachment means.

15. The knee bracing method of claim 14 comprising the steps of:
extending the anchor-to-brace tether structure from an anterior portion of the anchor structure along an anterior portion of the lower extremity; and
fastening the anchor-to-brace tether structure to an anterior portion of the knee brace via the tether-to-brace attachment means, thereby anteriorly tethering the knee brace to the anchor structure.

16. The knee bracing method of claim 14 comprising the step of fastening the anchor structure, the anchor-to-brace tether structure, and the tether-to-brace attachment means to an elastic pant construction, the elastic pant construction for enhancing control of said knee brace displacement.

17. The knee bracing method of claim 16 comprising the steps of:
extending the elastic pant construction in a layer intermediate the anchor-to-brace tether structure and the tether-to-brace attachment means for elastically fastening the anchor-to-brace tether structure to the tether-to-brace attachment means;
enabling a range of brace-to-extremity displacement via the elastically fastened anchor-to-brace tether structure and tether-to-brace attachment means; and
maintaining anatomical alignment of the knee brace axis with a variable flexion-extension axis of the outfitted lower extremity within said range of brace-to-extremity displacement.

18. The knee bracing method of claim 16 comprising the step of encircling a user's waist with the anchor structure for fixing the anchor structure in the transverse anchor plane.

19. The knee bracing method of claim 17 comprising the step of defining a tether-to-pant junction region during the step of extending the elastic pant construction in a layer intermediate the anchor-to-brace tether structure and the tether-to-brace attachment means, the area of restrained elasticity having reduced elasticity relative to the elastic material of the pant construction for simultaneously enabling both knee brace displacement control and pant construction comfort.

20. The knee bracing method of claim 17 comprising the step of removably fastening the tether-to-brace attachment means to the knee brace, the tether-to-brace attachment means being defined by a first select hook-loop fastening structure, the first select hook-loop fastening structure being selectively matable with a second select hook-loop fastening structure outfitted upon the knee brace, the first and second select hook-loop fastening structures being selected from the group consisting of hook type fastening structure and loop type fastening structure.
